(19) United States
(12) Patent Application Publication
(10) Pub. No.: US 2024/0062354 A1
(43) Pub. Date: Feb. 22, 2024

(54) IMAGE PROCESSING APPARATUS, IMAGE PROCESSING SYSTEM AND IMAGE PROCESSING METHOD

(71) Applicants: Canon Medical Systems Corporation, Otawara-shi (JP); CANON KABUSHIKI KAISHA, Tokyo (JP)

(72) Inventor: Shinya SUGIYAMA, Nasushiobara (JP)

(73) Assignees: Canon Medical Systems Corporation, Otawara-shi (JP); CANON KABUSHIKI KAISHA, Tokyo (JP)

(21) Appl. No.: 18/450,495

(22) Filed: Aug. 16, 2023

(30) Foreign Application Priority Data
Aug. 18, 2022 (JP) 2022-130658

(51) Int. Cl. G06T 7/00; G06T 7/246; C12M 1/34

(52) U.S. Cl. CPC G06T 7/0002; G06T 7/248; C12M 41/36; G06T 2207/10016; G06T 2207/30024

(57) ABSTRACT

According to one embodiment, an image processing apparatus includes processing circuitry. The processing circuitry specifies, based on a time-sequential photography image relating to a colony including a plurality of cultured cells, a growth direction of the colony, and a growth degree of the colony in the growth direction. The processing circuitry generates, based on the photography image, a growth image representing the growth direction and the growth degree of the colony.

### CROSS-REFERENCE TO RELATED APPLICATION

This application is based upon and claims the benefit of priority from prior Japanese Patent Application No. 2022-130658, filed Aug. 18, 2022, the entire contents of which are incorporated herein by reference.

### FIELD

Embodiments described herein relate generally to an image processing apparatus, an image processing system and an image processing method.

### BACKGROUND

Conventionally, there are proposed various methods for an observer to observe and evaluate a growth characteristic of an agglomerate (hereinafter referred to as “colony”) including a plurality of cultured cells. In one of such methods, a large number of images of the entirety of a colony are obtained with the passing of time, and a projection area of the colony at each culturing time is calculated, and a variation with time of the projection area is presented to an observer by a graph or the like.

However, the variation with time of the projection area of the colony, although being suitable for evaluating a growth characteristic in regard to the size of the colony, is not suitable for evaluating a growth characteristic in regard to the directionality of growth of the colony. In general, since the colony has different degrees of growth in individual growth directions, the colony has its shape varied dynamically. Accordingly, the growth characteristic of the colony cannot fully be evaluated by the evaluation of the area of the colony alone.

### BRIEF DESCRIPTION OF THE DRAWINGS

FIG. 1 is a diagram illustrating a configuration example of an image processing system according to a first embodiment.

FIG. 2 is a view illustrating an operation example of the image processing system according to the first embodiment.

FIG. 3 is a view illustrating an example of time-sequential photography images according to the first embodiment.

FIG. 4 is a view illustrating an example of a specifying method of a growth characteristic of a colony according to the first embodiment.

FIG. 5 is a view illustrating a display example of a growth image according to the first embodiment.

FIG. 6 is a view illustrating an operation example of the image processing system according to a second embodiment.

FIG. 7 is a view illustrating a specifying method of a growth characteristic of a cell group according to the second embodiment.

FIG. 8 is a view illustrating a display example of a growth image according to the second embodiment.

FIG. 9 is a view illustrating a display example of a growth image according to a modification.

### DETAILED DESCRIPTION

In general, according to one embodiment, an image processing apparatus includes processing circuitry. The processing circuitry specifies, based on a time-sequential photography image relating to a colony including a plurality of cultured cells, a growth direction of the colony, and a growth degree of the colony in the growth direction. The processing circuitry generates, based on the photography image, a growth image representing the growth direction and the growth degree of the colony.

Hereinafter, referring to the accompanying drawings, a description is given of an image processing apparatus, an image processing system and an image processing method according to embodiments. In the embodiments below, it is assumed that parts denoted by like reference signs perform the same operation, and an overlapping description is omitted unless where necessary.

### First Embodiment

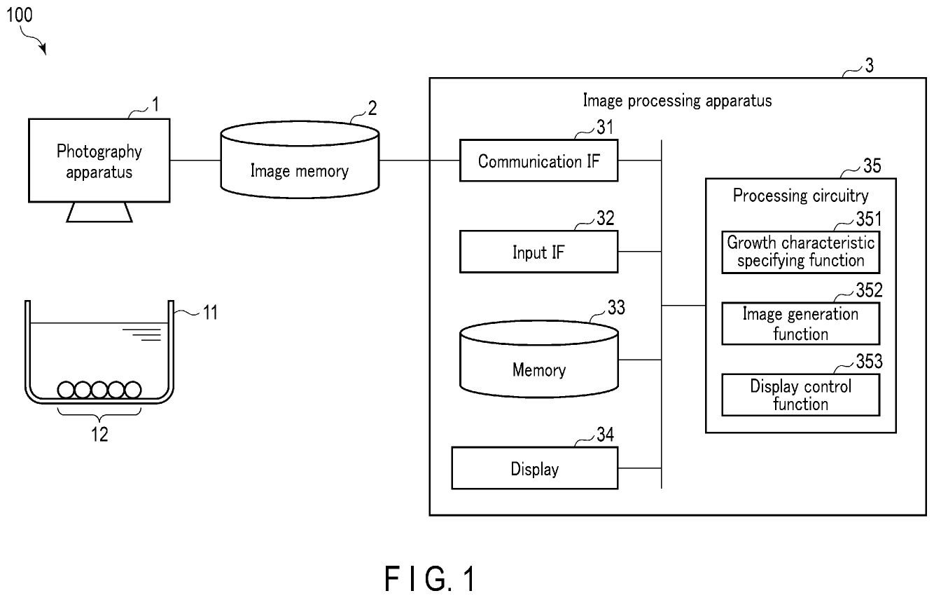

FIG. 1 is a diagram illustrating a configuration example of an image processing system 100 according to a first embodiment. The image processing system 100 is a system including a photography apparatus 1, an image memory 2 and an image processing apparatus 3. In the present embodiment, the photography apparatus 1 is communicably connected to the image memory 2, and the image memory 2 is communicably connected to the image processing apparatus 3. The communication method may be either wired communication or wireless communication.

The photography apparatus 1 is an apparatus that photographs a colony 12 including a plurality of cultured cells that are cultured in a culture container 11. Specifically, the photography apparatus 1 acquires time-sequential photography images relating to the colony 12, by photographing the colony 12 in a time-sequential manner. Preferably, the photography apparatus 1 is a CCD camera. Needless to say, the photography apparatus 1 may be various types of information processing apparatuses (for example, a smartphone, a tablet PC, a note PC) including photography mechanisms. In addition, the photography apparatus 1 is not limited to an optical camera such as a CCD camera, and may be an infrared camera or the like if cultured cells are recognizable on a photograph image. The photography apparatus 1 is an example of a photography unit. In the present embodiment, the photography apparatus 1 transfers an acquired photography image to the image memory 2.

In the present embodiment, the colony 12 is monolayer-cultured on a bottom surface of the culture container 11. Specifically, the colony 12 includes cultured cells having anchorage-dependency to a substrate. The photography apparatus 1 acquires time-sequential photography images of the entirety of the colony 12, by fixed-point photographing the colony 12 at predetermined time intervals in a direction perpendicular to the bottom surface of the culture container 11. Note that in the culture container 11, an environment (for example, a culture fluid, temperature, oxygen partial pressure, pH) suitable for culture of the colony 12 is maintained.

The image memory 2 is a device that stores photography images transferred from the photography apparatus 1. The image memory 2 includes, as a hardware resource, a semiconductor memory element such as a RAM (Random Access Memory). Note that the image memory 2 may be a drive unit that reads and writes information from and to a storage medium such as a magnetic disc (for example, a floppy (trademark) disc, a hard disk), a magneto-optical disc, an optical disc (for example, CD, DVD, Blu-ray (trademark)), or a flash memory (for example, a USB flash memory, a memory card, SSD). The image memory 2 is an example of a storage unit. In the present embodiment, the image memory 2 transfers stored photography images to the image processing apparatus 3. At this time, the image memory 2 may transfer a photography image to the image processing apparatus 3 in accordance with a request for the photography image from the image processing apparatus 3.

The image processing apparatus 3 is an apparatus that executes various image processes for the photography image transferred from the image memory 2. The image processing apparatus 3 includes, as structural elements, a communication IF 31, an input IF 32, a memory 33, a display 34, and processing circuitry 35. These structural elements are communicably connected via a bus (BUS) that is a common signal transmission path. Each structural element may be implemented by an individual hardware resource. Alternatively, at least two of the structural elements may be implemented by one hardware resource. Note that “IF” means “interface”.

The communication IF 31 communicates various kinds of information between the image memory 2 and the image processing apparatus 3. The communication IF 31 is an example of a communication unit. In the present embodiment, the communication IF 31 receives a photography image from the image memory 2, and transfers the received photography image to the memory 33 or the processing circuitry 35.

The input IF 32 receives various inputs from a user (for example, an observer) that uses the image processing apparatus 3, converts the received input to an electric signal, and outputs the electric signal to the processing circuitry 35. The input IF 32 may be various operating components operated by the user (for example, a mouse, a keyboard, a trackball, a switch, a button, a joystick, a touch pad, and a touch-panel display). The input IF 32 is an example of an input unit. In the present embodiment, the input IF 32 may receive, from the user, an input indicating that the generation of an image (hereinafter referred to as “growth image”) representing a growth characteristic of the colony 12 is started. The growth image represents the growth characteristic of the colony 12 by a coordinate system different from the photography image. Note that the “growth characteristic” means a growth direction or a growth degree of the colony 12. In addition, the growth direction of the colony 12 is a direction in which cultured cells constituting the colony 12 grow, and the growth degree of the colony 12 is a degree by which the cultured cells constituting the colony 12 grow.

The memory 33 stores various data and programs used by the processing circuitry 35. The structure of the memory 33 is the same as the structure of the image memory 2. The memory 33 is an example of the storage unit. In the present embodiment, the memory 33 stores photography images transferred from the communication IF 31, and growth images of the colony 12 generated by the processing circuitry 35.

The display 34 displays various data stored in the memory 33, and various data generated by the processing circuitry 35. As the display 34, use can be made of a freely selected display, such as a cathode ray tube (CRT) display, a liquid crystal display (LCD), a plasma display, or an organic EL (Electro-Luminescence) display (OELD). The display 34 is an example of a display unit. In the present embodiment, the display 34 displays a photography image stored in the memory 33, and a growth image of the colony 12 generated by the processing circuitry 35. The displayed image is viewed by the user.

The processing circuitry 35 controls the operation of the entirety of the image processing apparatus 3. The processing circuitry 35 includes at least one processor. The term “processor” means circuitry such as a CPU (Central Processing Unit), a GPU (Graphics Processing Unit), an application specific integrated circuit (ASIC), or a programmable logic device (for example, a simple programmable logic device (SPLD), a complex programmable logic device (CPLD) or a field programmable gate array (FPGA)). In a case where the processor is a CPU, the processor reads and executes programs stored in the memory 33, thereby implementing the functions corresponding to the programs. On the other hand, in a case where the processor is an ASIC, the respective functions are directly incorporated in the circuitry of the processor as logic circuitry, instead of the programs being stored in the memory 33. The processor may be constructed as single circuitry, or may be constructed by combining a plurality of independent circuits. The processing circuitry 35 is an example of a processing unit. In the present embodiment, the processing circuitry 35 implements the respective functions (for example, a growth characteristic specifying function 351, an image generation function 352 and a display control function 353).

The growth characteristic specifying function 351 specifies a growth direction of the colony 12, and a growth degree of the colony 12 in the growth direction 12, based on a time-sequential photography image relating to the colony 12 including cultured cells. For example, the growth characteristic specifying function 351 specifies the growth direction and growth degree of the colony 12, in a polar coordinate system defined by an angle around a reference point of the photography image, and a distance from the reference point at the angle. Specifically, in the polar coordinate system, the growth characteristic specifying function 351 specifies the growth direction of the colony 12, based on the angle at which each cultured cell constituting the colony 12 is located, and specifies the growth degree of the colony 12, based on the distance from the reference point to each cell. The growth characteristic specifying function 351 is an example of a growth characteristic specifying unit.

The image generation function 352 generates, based on the photography image of the colony 12, a growth image representing the growth direction and growth degree of the colony 12. For example, the image generation function 352 generates a growth image representing the growth direction and growth degree of the colony 12 in an orthogonal coordinate system in which an angle around a reference point of the photography image is indicated on one axis and a distance from the reference point at the angle is indicated on the other axis. Specifically, the image generation function 352 generates a growth image representing the growth direction and growth degree of the colony 12 at each time point in the orthogonal coordinate system by a line corresponding to each time point. The image generation function 352 is an example of an image generation unit.

The display control function 353 causes the display 34 to display various images. For example, the display control function 353 causes the display 34 to display the growth image of the colony 12. Note that the display control function 353 may cause the display 34 to display photography images used in generating the growth image.

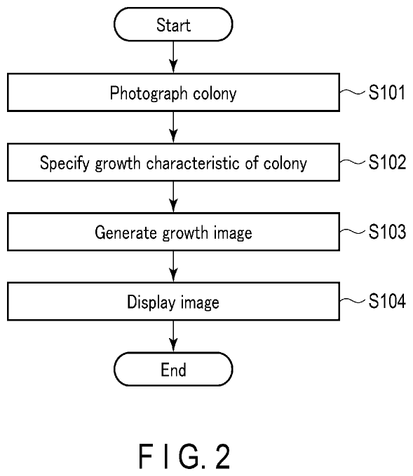

FIG. 2 is a view illustrating an operation example of the image processing system 100 according to the first embodiment. The present operation example may be started in response to an input by the user indicating that the generation of the growth image of the colony 12 is started.

(Step S101) To begin with, the image processing system 100 photographs the colony 12 by the photography apparatus 1. Specifically, the photography apparatus 1 acquires time-sequential photography images by photographing the colony 12 in a time-sequential manner. As described above, the acquired photography images are transferred to the image memory 2, and then transferred to the image processing apparatus 3.

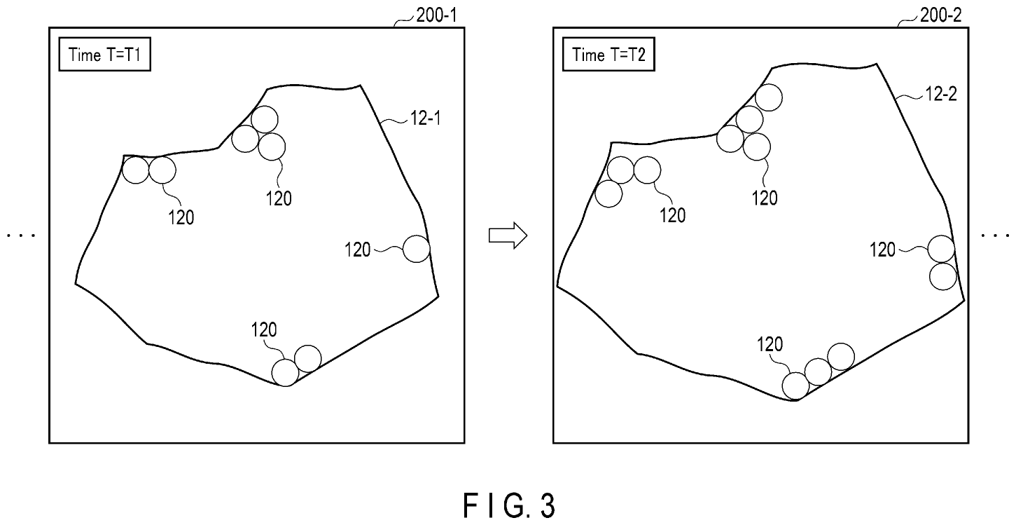

FIG. 3 is a view illustrating an example of time-sequential photography images 200 according to the first embodiment. Here, as the time-sequential photography images 200, a photography image 200-1 and a photography image 200-2 are illustrated, the photography image 200-1 representing an entire image of a colony 12-1 in a case where a time T from the start of photography is T1 (T=T1), and the photography image 200-2 representing an entire image of a colony 12-2 in a case where the time T is T2 (T=T2). Note that after the photography image 200-1 is acquired, the photography image 200-2 is acquired (i.e., T1<T2). The photography image 200-1 may be an image at the time of the start of photography, or may be an image after the passing of a predetermined time from the start of photography.

The photography image 200-1 displays the colony 12-1 including a plurality of cultured cells 120. For the purpose of convenience of the description, some cultured cells 120, among all cultured cells 120 constituting the colony 12-1, and an outline of the colony 12-1, are illustrated. The outline of the colony 12-1 is an outline defined by the cultured cells 120 located on an outermost edge of the colony 12-1. The colony 12-1 has such an irregular shape that a part thereof (an upper left part) is recessed inward. In the present embodiment, it is assumed that the colony 12-1 grows at a uniform velocity in each radial direction from the center thereof as a starting point. After the passing of a predetermined time, the colony 12-1 grows to the colony 12-2 displayed on the photography image 200-2.

The photography image 200-2 displays the colony 12-2 including cultured cells 120. Since the colony 12-2 includes a greater number of cultured cells 120 than the colony 12-1, the area of the colony 12-2 is greater than the area of the colony 12-1. Note that the growth of the colony 12 includes a proliferation process of cultured cells 120 and a growth process of each of the proliferated cultured cells 120. In general, cultured cells 120, which are being divided, are located on the outermost edge of the colony 12, and cultured cells 120, which are grown, are located in the inside of the colony 12. There is a case where the cultured cells 120, which located on the outermost edge of the colony 12, are larger than the cultured cells 120 located in the inside of the colony 12.

(Step S102) Next, the image processing system 100 specifies the growth characteristic of the colony 12 by the image processing apparatus 3. Specifically, the processing circuitry 35 of the image processing apparatus 3 specifies, by the growth characteristic specifying function 351, the growth direction of the colony 12 and the growth degree of the colony 12 in the growth direction, based on the time-sequential photography images 200.

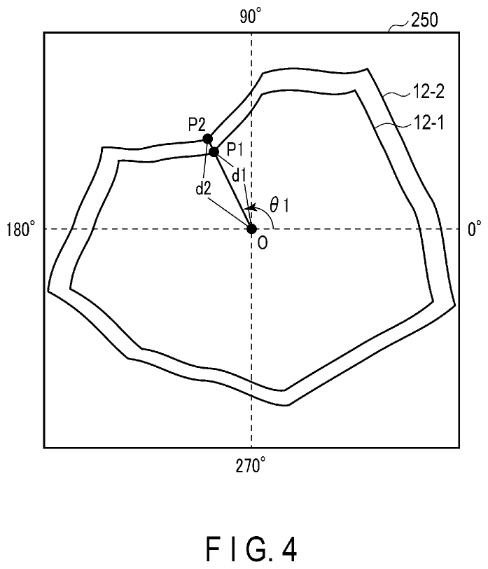

FIG. 4 is a view illustrating an example of a specifying method of a growth characteristic of the colony 12 according to the first embodiment. To begin with, the image processing apparatus 3 generates a photography image 250 in which the photography image 200-1 and photography image 200-2 are composited. At this time, the image processing apparatus 3 may composite both the photography image 200-1 and the photography image 200-2 such that a reference point (for example, the center of gravity, the origin of cell tracking) of the colony 12-1 in the photography image 200-1 is aligned with a reference point of the colony 12-2 in the photography image 200-2. For the alignment between the reference points, a known method is applicable. Note that, instead of the reference point, a freely chosen line on the colony 12 may be used as a reference for alignment. It suffices that the reference point exists on the photography image 200, and the reference point may be set in the inside of the colony 12 or may be set outside the colony 12.

Next, the image processing apparatus 3 sets, on the photography image 250, a polar coordinate system having as an origin O the above-described aligned reference point. Specifically, the image processing apparatus 3 sets one axis (X axis) passing through the origin O on the photography image 250, defines one direction of this axis as a 0° direction, and defines the other direction as a 1800 direction. In addition, the image processing apparatus 3 sets another axis (Y axis) that passes through the origin O and is perpendicular to the X axis, defines one direction of this axis as a 90° direction, and defines the other direction as a 2700 direction. Thereby, the image processing apparatus 3 can express a freely chosen position on the photography image 250 by polar coordinates (d, θ) that are a set of a distance d from the origin O to this position and an angle θ from the 0° direction of the X axis.

Subsequently, the image processing apparatus 3 specifies the growth direction and growth degree of the colony 12 in the above-described polar coordinate system. Specifically, the image processing apparatus 3 specifies the growth direction of the colony 12 by the angle θ at which each cultured cell 120 constituting the colony 12 is located, and specifies the growth degree of the colony 12 by the distance d from the reference point. In one example, attention is paid to a point P1 that is present on the outline of the colony 12-1 and is located in a most inwardly recessed portion (upper left portion) of the colony 12-1. The point P1 is, in other words, a position of a predetermined cultured cell 120 that forms the outline of the colony 12-1. In this case, the image processing apparatus 3 specifies the growth direction and growth degree of the colony 12 in a straight direction passing through the origin O and point P1.

By analyzing the photography image 250, the image processing apparatus 3 calculates a distance “d1” between the point P1 and the origin O and an angle “θ1” around the origin O. Next, by executing similar image analysis with respect to a point P2 that is present on a straight line connecting the origin O and point P1 and is located on the outline of the colony 12-2, the image processing apparatus 3 calculates a distance “d2” (d1<d2) between the point P2 and the origin O. Thereby, the image processing apparatus 3 specifies the angle “θ1” as the growth direction of the colony 12 at the point P1, and specifies the distance “d1” as the growth degree of the colony 12 in the growth direction. Similarly, the image processing apparatus 3 specifies the angle “θ1” as the growth direction of the colony 12 at the point P2, and specifies the distance “d2” as the growth degree of the colony 12 in the growth direction. Note that a machine learning model, which is trained in advance, may be applied to the image analysis of the photography image 250.

Subsequently, the image processing apparatus 3 repeats an operation similar to the above, in regard to each point located on the outline of the colony 12. Thereby, the growth degree of the colony 12 at each angle θ is specified by the distance d. Note that the growth direction and growth degree of the colony 12 can be specified by a vector.

(Step S103) Subsequently, the image processing system 100 generates a growth image of the colony 12 by the image processing apparatus 3. Specifically, the processing circuitry 35 of the image processing apparatus 3 generates a growth image of the colony 12 by the image generation function 352, based on the photography images 200 and the growth direction and growth degree of the colony 12. An image conversion by machine learning or the like may be applied to the generation of the growth image.

(Step S104) At last, the image processing system 100 displays the growth image of the colony 12 by the image processing apparatus 3. Specifically, the processing circuitry 35 of the image processing apparatus 3 causes the display 34 to display the growth image by the display control function 353. Thereby, the user can view the growth image displayed on the display 34. After the execution of this step, the image processing system 100 terminates the sequence of the process.

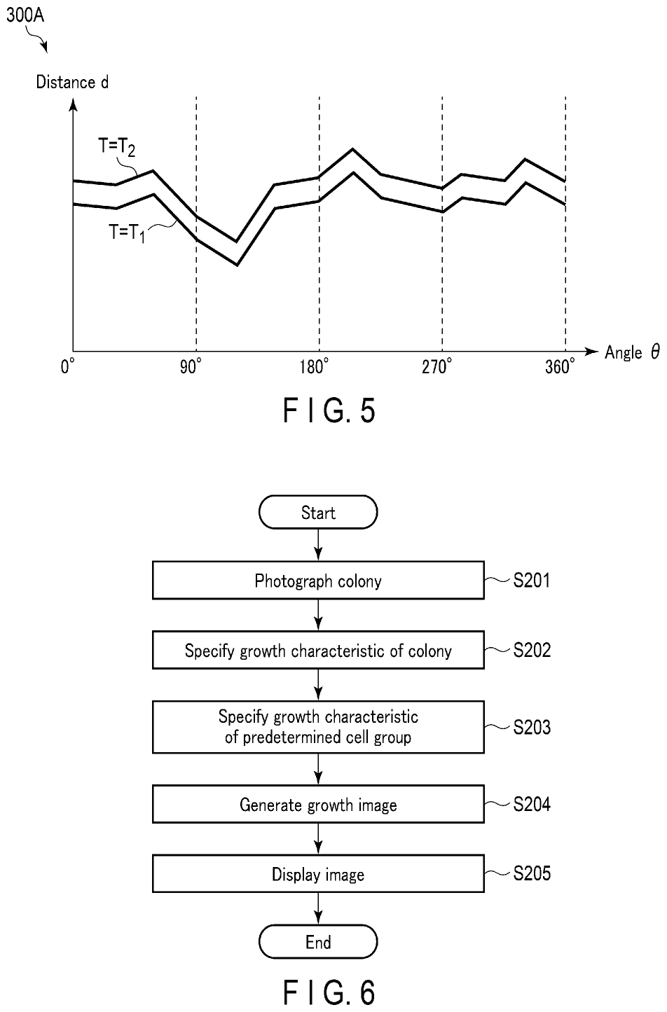

FIG. 5 is a view illustrating a display example of a growth image 300A according to the first embodiment. The growth image 300A represents the growth direction and growth degree of the colony 12 in an orthogonal coordinate system in which an angle θ is indicated on the abscissa axis and a distance d is indicated on the ordinate axis. In other words, the growth image 300A is a diagram in which the photography image 250 is developed in the direction of the angle θ. The growth image 300A displays a line (T=T1) indicating the growth degree of the colony 12-1 in the photography image 200-1, and a line (T=T2) indicating the growth degree of the colony 12-2 in the photography image 200-2.

According to the line indicating the growth degree of the colony 12-1, it is understood that, in regard to the colony 12-1, the distance d is shortest in a range of 90° to 180° of the angle θ, and the distance d is longest in a range of 180° to 270° of the angle θ. In other words, the colony 12-1 is most inwardly recessed in the former range of the angle θ, and the colony 12-1 is most outwardly projected in the latter range of the angle θ. In this manner, by confirming the line indicating the growth degree of the colony 12-1, the user can understand the shape of the colony 12-1. Similarly, by confirming the line indicating the growth degree of the colony 12-2, the user can understand the shape of the colony 12-2.

In addition, according to the line indicating the growth degree of the colony 12-2, it is understood that the colony 12-2 has a similar shape to the shape of the colony 12-1. In regard to the colony 12-2, however, the distance d is greater than in the colony 12-1 at all angles θ (i.e., the area increases). By comparing the line indicating the growth degree of the colony 12-1 and the line indicating the growth degree of the colony 12-2, the user can understand a variation with time of the shape of the colony 12, for example, in what range of the angle θ the colony 12 has particularly grown. In other words, a difference between the line indicating the growth degree of the colony 12-1 and the line indicating the growth degree of the colony line 12-2 (a region between both lines) corresponds to an amount of the growth of the colony 12-1 during a period from time T1 to T2. This difference may be expressed by being painted by a predetermined color or pattern on the growth image 300A.

Although the two lines are displayed on the growth image 300A according to the present embodiment, a plurality of lines are displayed on the growth image 300A in accordance with the number of photography images 200. In this case, a plurality of lines corresponding to time points, at which the photography images 200 are acquired, are displayed by being overlapped in the direction of the distance d. Thereby, a variation with time relating to the shape of the colony 12 is presented.

Note that a left end (a 0° position of the angle θ) and a right end (a 360° position of the angle θ) of a graph display area of the growth image 300A may be connected. In addition, in accordance with a scroll operation in the left-and-right by the user on the growth image 300A, the graph display area of the growth image 300A may be rotated in the left-and-right direction and displayed. Besides, the photography image 250 illustrated in FIG. 4, together with the growth image 300A, may be displayed on the same screen. Thereby, the correspondence relation between the angle θ in the growth image 300A and the angle θ in the photography image 250 is presented.

The image processing system 100 according to the first embodiment has been described above. The image processing system 100 according to the present embodiment specifies the growth direction of the colony 12 and the growth degree of the colony 12 in the growth direction, based on the time-sequential photography images 200 relating to the colony 12 including cultured cells. Based on the photography images 200, the image processing system 100 generates and displays the growth image 300A representing the growth direction and growth degree of the colony 12.

By confirming the displayed growth image 300A, the user can evaluate the growth degree in each growth direction of the colony 12. From another viewpoint, the image processing system 100 can support the user in evaluating the growth degree in each growth direction of the colony 12.

### Second Embodiment

The image processing system 100 according to the first embodiment presents the growth characteristic of the entirety of the colony 12. In addition to this, an image processing system 100 according to a second embodiment further presents a growth characteristic of a cell group that proliferates from a predetermined cultured cell included in the colony 12. The configuration of the image processing system 100 according to the second embodiment is similar to the configuration in the first embodiment.

FIG. 6 is a view illustrating an operation example of the image processing system 100 according to the second embodiment. Like the first embodiment, the present operation example may be started in response to an input by the user indicating that the generation of the growth image of the colony 12 is started.

(Step S201) To begin with, the image processing system 100 photographs the colony 12 by the photography apparatus 1. Step S201 is similar to step S101.

(Step S202) Next, the image processing system 100 specifies the growth characteristic of the colony 12 by the image processing apparatus 3. Step S202 is similar to step S102.

(Step S203) Subsequently, the image processing system 100 specifies the growth characteristic of a predetermined cell group included in the colony 12 by the image processing apparatus 3. Specifically, the processing circuitry 35 of the image processing apparatus 3 specifies, by the growth characteristic specifying function 351, the growth direction of a cell group that proliferates from a predetermined cultured cell included in the colony 12, and the growth degree of the cell group in the growth direction. Note that step S203 may be executed before step S202.

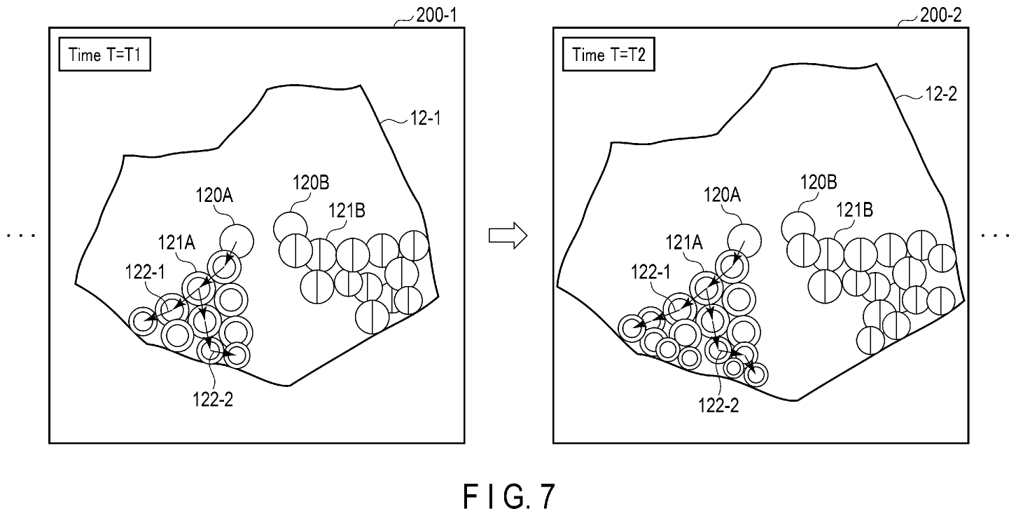

FIG. 7 is a view illustrating a specifying method of a growth characteristic of a cell group according to the second embodiment. To begin with, the image processing apparatus 3 executes tracking analysis of predetermined cultured cells included in the colony 12 with respect to each of the photography images 200. The tracking analysis is a method of observing a division, motion or the like of a predetermined cell with the passing of time, and a known method is applicable. For example, the image processing apparatus 3 executes tracking analysis of two cultured cells 120A and 120B, thereby specifying cell groups 121A and 121B that proliferate from the cultured cells 120A and 120B, respectively. Here, the cell group 121A is expressed by a mark of a double circle, and the cell group 121B is expressed by a mark of a combination of two semicircles. In addition, as regards the cell group 121A, growth directions 122-1 and 122-2 of the cell group 121A are specified. Note that the cultured cell 120A may be included in the cell group 121A, and the cultured cell 120B may be included in the cell group 121B. Needless to say, growth directions may similarly be specified in regard to the other cell group 121B.

The growth directions 122-1 and 122-2 are paths of series of cells proliferating from the cultured cell 120A, and are parts of cell lineages starting from the cultured cell 120A. The growth directions 122-1 and 122-2 have a Y-shaped branch structure and have a common path before branching, but have different paths after branching. The growth directions 122-1 and 122-2 are expressed by arrows along the directions of proliferation of the cultured cell 120A. From the directions of the arrows, it is understood that the cultured cell 120A has continued to proliferate from a central portion toward a marginal portion of the colony 12-1.

Note that, using a method similar to the method in FIG. 4, the image processing apparatus 3 may specify the growth directions and growth degrees of the cell groups 121A and 121B, in a polar coordinate system defined by an angle around a reference point of the photography image 200, and a distance from the reference point at the angle. Thereby, the arrows indicating the growth directions 122-1 and 122-2 of the cell group 121A are expressed in the polar coordinate system.

(Step S204) Subsequently, the image processing system 100 generates a growth image of the colony 12 by the image processing apparatus 3. Step S204 is substantially similar to step S103. In step S204, however, the processing circuitry 35 of the image processing apparatus 3 generates a growth image of the colony 12, which further represents the growth directions and growth degrees of the cell group, which are specified in step S203.

(Step S205) At last, the image processing system 100 displays the growth image of the colony 12 by the image processing apparatus 3. Step S205 is similar to step S104. After the execution of this step, the image processing system 100 terminates the sequence of the process.

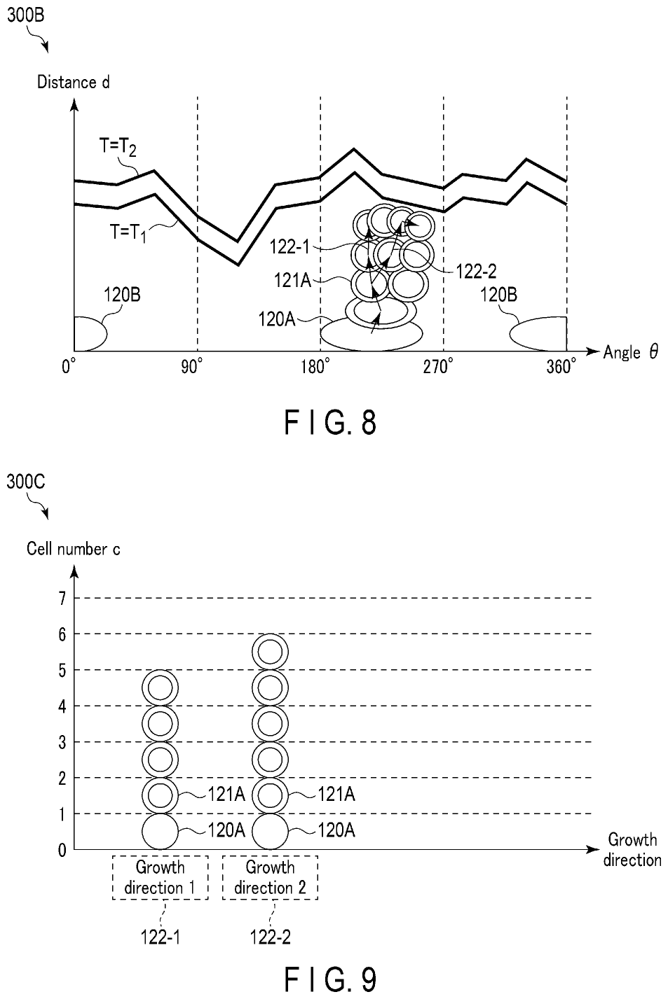

FIG. 8 is a view illustrating a display example of a growth image 300B according to the second embodiment. The growth image 300B is substantially similar to the growth image 300A according to the first embodiment. The growth image 300B, however, represents the growth directions and growth degrees of the cell group at each time point by the arrows corresponding to each time point. In particular, the growth directions 122-1 and 122-2 of the cell group 121A are expressed by arrows. The presence and absence of the display of the arrows may be switched in accordance with an operation by the user.

In addition, in the growth image 300B, an image relating to the cultured cells 120A and 120B is composited and displayed. Furthermore, an image relating to the cell group 121A proliferating from the cultured cell 120A is displayed. Both of the images are generated by extracting, by the image processing apparatus 3, an image area including the cultured cells 120A and 120B and an image area including the cell group 121A, from the photography image, and processing the extracted image areas. At this time, since the cultured cells 120A and 120B near the reference point of the photography image 200 have a relatively large range of the angle θ occupied in the angle θ direction, the cultured cells 120A and 120B are displayed by being greatly extended in the angle θ direction. On the other hand, since the cultured cells of the cell group 121A, which are relatively remote from the reference point of the photography image 200, have a relatively small range of the angle θ occupied in the angle θ direction, the cultured cells of the cell group 121A are displayed without being greatly extended in the angle θ direction.

Note that in the growth image 300B, instead of the image of cells, a pattern (mark) in which the image of cells is processed may be composited and displayed. The pattern may be a freely chosen figure (for example, a circle, crisscross (x), triangle, or rectangle).

Although the growth directions and growth degrees of the cell group 121A starting from the cultured cell 120A are displayed by the arrows in the growth image 300B, the embodiment is not limited to this. The user may select the image of the other cultured cell 120B in the growth image 300B, and thereby the growth directions and growth degrees of the cell group 121B starting from the selected cultured cell 120B may be displayed in a switched manner. According to this display mode, the user can confirm a desired cultured cell, and the growth directions and growth degrees of a cell group starting from this cultured cell. Needless to say, the growth directions and growth degrees of both of the cell groups 121A and 121B may be displayed at the same time.

The image processing system 100 according to the second embodiment has been described above. In addition to the operation according to the first embodiment, the image processing system 100 according to the second embodiment further specifies the growth directions of the cell group proliferating from a predetermined cultured cell included in the colony 12, and the growth degrees of the cell group in the growth direction, based on the time-sequential photography images relating to the colony 12 including cultured cells. Based on the photography images, the image processing system 100 generates and displays the growth image 300B that further represents the growth directions and growth degrees of the cell group.

By confirming the displayed growth image 300B, the user can further evaluate the growth degree in each growth direction of the cell group included in the colony 12, in addition to the growth degree in each growth direction of the colony 12. From another viewpoint, the image processing system 100 can support the user in evaluating the growth degree in each growth direction of the cell group, in addition to the growth degree in each growth direction of the colony 12.

### (Modification)

FIG. 9 is a view illustrating a display example of a growth image 300C according to a modification. The growth image 300C represents the growth degree of the cell group 121A in each of the growth directions 122-1 and 122-2 of the cell group 121A by a cell number c of cells existing in each growth direction. The growth image 300C is a graph in which “growth direction” is indicated on the abscissa axis and “cell number c” is indicated on the ordinate axis. Note that the cell number c may be specified by using the result of the above-described tracking analysis.

For example, in “growth direction 1” corresponding to the growth direction 122-1, the cultured cell 120A that is the starting point, and four cultured cells included in the cell group 121A proliferating from the cultured cell 120A, are stacked and displayed in the ordinate-axis direction. Similarly, in “growth direction 2” corresponding to the growth direction 122-2, the cultured cell 120A that is the starting point, and five cultured cells included in the cell group 121A proliferating from the cultured cell 120A, are stacked and displayed in the ordinate-axis direction. Needless to say, as regards the cell group 121B proliferating from the cultured cell 120B, a similar display as described above may be made.

By confirming the growth image 300C, the user can evaluate the growth degree in each growth direction of the cell group by the cell number in each growth direction.

As described above, the image processing apparatus 3 specifies the growth direction and growth degree of the colony 12 in the photography image 200 in the polar coordinate system that is set in the photography image 200, and generates the growth image in which the specified growth direction and growth degree of the colony 12 are expressed by the orthogonal coordinate system. Aside from this, the image processing apparatus 3 may generate a growth image (i.e., a polar coordinate graph) in which the specified growth direction and growth degree of the colony 12 are expressed by the polar coordinate system. In this case, the image processing apparatus 3 may plot the growth degrees in the respective growth directions of the colony 12 on a circular grid, and may display a line connecting each plot. By confirming the line corresponding to each time point, the user can understand a variation with time of the shape of the colony 12.

In addition, in order to specify the growth direction and growth degree of the colony 12, the image processing apparatus 3 may use any one of (A) a minimum distance to a margin (outline) of the colony 12, (B) a path (cell tracking path) of tracking analysis of cultured cells 120 included in the colony 12 and a statistical tendency thereof, (C) a conversion by machine learning or the like, (D) a growth degree (time-sequential) vector of the colony 12, and (E) a contour line of the colony 12.

According to at least one of the above-described embodiments, the directionality of growth of a colony can be presented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising processing circuitry, the processing circuitry being configured to:
specify, based on a time-sequential photography image relating to a colony including a plurality of cultured cells, a growth direction of the colony, and a growth degree of the colony in the growth direction; and
generate, based on the photography image, a growth image representing the growth direction and the growth degree of the colony.

2. The image processing apparatus of claim 1, wherein the processing circuitry specifies the growth direction and the growth degree of the colony, in a polar coordinate system defined by an angle around a reference point of the photography image, and a distance from the reference point at the angle.

3. The image processing apparatus of claim 2, wherein in the polar coordinate system, the processing circuitry specifies the growth direction of the colony, based on an angle at which each of the cultured cells constituting the colony is located, and specifies the growth degree of the colony, based on a distance from the reference point to each of the cultured cells.

4. The image processing apparatus of claim 2, wherein the processing circuitry generates the growth image that represents the growth direction and the growth degree of the colony in an orthogonal coordinate system in which the angle is indicated on one axis and the distance from the reference point at the angle is indicated on the other axis.

5. The image processing apparatus of claim 4, wherein the processing circuitry generates the growth image that represents the growth direction and the growth degree of the colony at each of time points in the orthogonal coordinate system by a line corresponding to each time point.

6. The image processing apparatus of claim 1, wherein the processing circuitry is configured to:
further specify, based on the photography image, a growth direction of a cell group proliferating from a predetermined cultured cell included in the colony, and a growth degree of the cell group in the growth direction; and
generate, based on the photography image, the growth image further representing the growth direction and the growth degree of the cell group.

7. The image processing apparatus of claim 6, wherein the processing circuitry specifies the growth direction and the growth degree of the cell group by executing tracking analysis of the predetermined cultured cell.

8. The image processing apparatus of claim 6, wherein the processing circuitry specifies the growth direction and the growth degree of the cell group, in a polar coordinate system defined by an angle around a reference point of the photography image, and a distance from the reference point at the angle.

9. The image processing apparatus of claim 8, wherein the processing circuitry generates the growth image that represents the growth direction and the growth degree of the cell group in an orthogonal coordinate system in which the angle is indicated on one axis and a distance from the reference point at the angle is indicated on the other axis.

10. The image processing apparatus of claim 9, wherein the processing circuitry generates the growth image that represents the growth direction and the growth degree of the cell group at each of time points in the orthogonal coordinate system by an arrow corresponding to each time point.

11. The image processing apparatus of claim 6, wherein the processing circuitry extracts an image area including the cell group from the photography image, and generates the growth image in which the image area or a pattern obtained by processing the image area is composited.

12. An image processing system comprising:
a photography apparatus configured to photograph a colony including a plurality of cultured cells in a time-sequential manner, thereby acquiring a time-sequential photography image relating to the colony;
an image memory configured to store the photography image;
processing circuitry configured to
specify, based on the photography image, a growth direction of the colony, and a growth degree of the colony in the growth direction, and
generate, based on the photography image, a growth image representing the growth direction and the growth degree of the colony; and
a display configured to display the growth image.

13. An image processing method comprising:
specifying, based on a time-sequential photography image relating to a colony including a plurality of cultured cells, a growth direction of the colony, and a growth degree of the colony in the growth direction; and
generating, based on the photography image, a growth image representing the growth direction and the growth degree of the colony.

14. The image processing method of claim 13, further comprising specifying the growth direction and the growth degree of the colony, in a polar coordinate system defined by an angle around a reference point of the photography image, and a distance from the reference point at the angle.

15. The image processing method of claim 14, further comprising:
specifying, in the polar coordinate system, the growth direction of the colony, based on an angle at which each of the cultured cells constituting the colony is located; and
specifying the growth degree of the colony, based on a distance from the reference point to each of the cultured cells.

16. The image processing method of claim 14, further comprising generating the growth image that represents the growth direction and the growth degree of the colony in an orthogonal coordinate system in which the angle is indicated on one axis and the distance from the reference point at the angle is indicated on the other axis.

17. The image processing method of claim 16, further comprising generating the growth image that represents the growth direction and the growth degree of the colony at each of time points in the orthogonal coordinate system by a line corresponding to each time point.

18. The image processing method of claim 13, further comprising:
further specifying, based on the photography image, a growth direction of a cell group proliferating from a predetermined cultured cell included in the colony, and a growth degree of the cell group in the growth direction; and
generating, based on the photography image, the growth image further representing the growth direction and the growth degree of the cell group.

19. The image processing method of claim 18, further comprising specifying the growth direction and the growth degree of the cell group by executing tracking analysis of the predetermined cultured cell.

20. The image processing method of claim 18, further comprising specifying the growth direction and the growth degree of the cell group, in a polar coordinate system defined by an angle around a reference point of the photography image, and a distance from the reference point at the angle.
